(19) United States
(12) Patent Application Publication
(10) Pub. No.: US 2009/0163899 A1
(43) Pub. Date: Jun. 25, 2009

(54) SWAGED OPTICAL FIBER CATHETER TIPS AND METHODS OF MAKING AND USING

(75) Inventors: George W. Burton, Colorado Springs, CO (US); Holly A. Scott, Colorado Springs, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Spectranetics, Colorado Springs, CO (US)

(21) Appl. No.: 11/962,998

(22) Filed: Dec. 21, 2007

(51) Int. Cl. A61B 18/20; A61B 18/18; A61L 33/02

(52) U.S. Cl. 606/15; 427/2.1

(57) ABSTRACT

A fiber optic unit to ablate tissue with light is described. The unit may include a bundle of optical fibers having a bundle proximal end adaptable to a light source, and a hardened bundle distal end though which the light exits to reach the tissue. The unit may also include hard materials, such as metal or glass, formed around the distal ends of each of the optical fibers. The hard coatings may be fused or swaged to bond the distal ends of the optical fibers together.

### BACKGROUND OF THE INVENTION

Tissue ablation with pulses of light energy can be used to treat a variety of ailments. For example, the catheters can be used to ablate vascular occlusions that restrict the flow of blood to tissue and organs. When these occlusions develop in vessels supplying blood to heart (e.g., coronary arteries and veins) they can cause heart attacks and angina. When they develop in vessels supplying blood to the brain (e.g., cerebral arteries and veins), they can cause strokes and other neurological problems. In tissue ablation, the pulses of light energy are used to disrupt these vascular occlusions to increase or reopen or the flow of blood through the vessel.

Tissue ablation methods to recanalize a blood vessel are minimally invasive and may include advancing a distal end of a light-guiding catheter to a position close to the occlusion. The light-guiding catheter typically has an optical fiber that can transmit light energy from a light source to the target tissue with minimal energy loss. For example, the catheter may be advanced through a patient's vasculature to the site of the occlusion and then an optical fiber may be advanced through a lumen inside the catheter to reach the occlusion. When the distal end of the fiber is in place, light pulses originating at a light source are sent through the fiber to irradiate the occlusion. A light source producing a high intensity/high energy light pulse can be highly effective at fragmenting soft tissue occlusions.

However, additional classes of occlusions such as chronic total occlusions (CTOs) have proven much harder to treat with tissue ablation. CTOs are generally calcified, fibrotic occlusions that are difficult to fragment with pulses of light energy. In response, the ablation power has been increased to more effectively disrupt these kinds of occlusions. However, attempts to increase the energy density of the light pulse have presented some technical challenges.

In order to efficiently transmit light pulses energy with increased energy density, the cross-sectional area of the optical fiber can be increased. However, for single fiber catheter systems a practical limit on the thickness of the fiber is quickly reached, because the fiber has to be capable of traveling through tortuous blood vessels to reach the target tissue. The thicker the optical fiber becomes the less flexible it becomes, making it more difficult to advance to the target.

Another approach is to divide the light pulse through a group of smaller diameter fibers that have coaxially aligned distal ends to deliver the pulse of light energy to the target tissue. Even when grouped together, the smaller fibers are more flexible than a single fiber of the same diameter and better able to navigate the bends and twists of a patient's vasculature. However, the bundled optical fibers need to be glued or bonded together at the distal tip in order to maintain position and ensure security in case of a fiber break. The presently favored bundling materials are biocompatible epoxy resins.

Unfortunately, these epoxies are not very durable under the acoustic shock conditions that are typical for Excimer laser ablation of hard, calcified target tissue (e.g., CTOs). In as little as 1000 light pulses, enough epoxy can etch from the distal tip of the light catheter to expose the distal edges and walls of the optical fibers. Exposed optical fibers are fragile and even slight force can fracture the fibers and significantly reduce the amount of light they can transmit to the target. Thus, there is a need for new ways to bundle the distal ends of optical fibers used in the light ablation of tissue. This an other issues are addressed by the present invention.

### BRIEF SUMMARY OF THE INVENTION

The present invention uses an optical fiber bundle with a hardened distal tip to ablate target tissue in a patient. The hardened distal tip uses materials that are harder and more durable than the epoxies used to bind individual optical fibers in conventionally bundled fiber optic tips. These materials may include metals, silicate glasses, and diamond like carbon (DLC) films, among other types of hard bonding materials. The added durability helps maintain the integrity of the tip when the fiber optic unit (e.g., a light-guiding catheter) is ablating hardened target tissue, such as a calcified chronic total occlusion (CTO). The acoustic shock that accompanies the ablating of the hardened tissue can etch the epoxy in less than 1000 light pulses. In contrast, a hardened tip using glass or metal bonding materials can still be largely intact after more than 62,000 light pulses. Thus, the hard bonded tips can be used to ablate hardened tissue for more light pulses and with less loss of mechanical integrity and transmission efficiency than conventional, epoxy bonded tips.

Embodiments of the invention include a fiber optic unit to ablate tissue with light. The unit may include a bundle of optical fibers having a bundle proximal end adaptable to a light source, and a bundle distal end though which the light exits to reach the tissue. The unit may also include hard material coatings (e.g., glass, ceramic, or metal coatings) formed around distal ends of each of the optical fibers, where the metal coatings are swaged to bond the distal ends of the optical fibers together.

Embodiments of the invention may also include methods of making an optical fiber bundle for tissue ablation having a hard bonded distal end. The methods may include providing a plurality of optical fibers that includes a light transmitting core and a polymeric coating, and stripping the polymeric coating from distal wall portions of the optical fiber. The methods may also include depositing a metal coating on the stripped distal wall portion of the optical fiber, and swaging the distal ends of the optical fibers together to form the metal bonded distal end of the bundle.

Embodiments of the invention may still further include methods to ablate target tissue with light. The methods may include providing a fiber optic unit that includes a bundle of optical fibers having a bundle proximal end adapted to a light source and a bundle distal end though which the light exits to reach the tissue. The hard coating (e.g., metal coating) formed around distal ends of each of the optical fibers are swaged to bond the distal ends of the optical fibers together. The methods may also include advancing the bundle's distal end to a position proximate to the target tissue, and transmitting the light through the optical fibers to ablate the target tissue.

Embodiments of the invention may yet still further include a fiber optic unit having a glass-fused distal tip to ablate tissue with light. The unit may include a bundle of optical fibers having a bundle proximal end adaptable to a light source, and a bundle distal end though which the light exits to reach the tissue. A glass coating may be formed around distal ends of each of the optical fibers, wherein the glass coatings are fused to bond the distal ends of the optical fibers together.

Embodiments of the invention may yet also include methods of making an optical fiber bundle for tissue ablation having a glass fused distal end. The methods may include providing a plurality of optical fibers, where each fiber includes a fused-silica light transmitting core surrounded with a polymeric coating. The method may further include stripping the polymeric coating from distal wall portions of the optical fiber, and heating the stripped distal ends of the optical fibers to fused them together into the glass fused distal end of the optical fiber bundle.

Embodiments of the invention may also include methods of making an optical fiber bundle for tissue ablation having a hardened distal end. The methods may include providing a plurality of optical fibers comprising a fused-silica light transmitting core surrounded with a polymeric coating. The methods may also include stripping the polymeric coating from distal wall portions of the optical fiber, and depositing a hardening material on the stripped distal ends of the optical fibers. The methods may still also include curing the deposited hardening material to form the hardened distal end of the optical fiber bundle.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components. In some instances, a sublabel is associated with a reference numeral and follows a hyphen to denote one of multiple similar components. When reference is made to a reference numeral without specification to an existing sublabel, it is intended to refer to all such multiple similar components.

FIG. 1 is a cross-sectional schematic drawing of a metal bonded distal end of an optical fiber bundle without an inner lumen (e.g., a guidewire lumen) according to embodiments of the invention;

FIG. 2 is a cross-sectional schematic drawing of a hardened material bonded distal end of an optical fiber bundle without an inner lumen according to embodiments of the invention;

FIG. 3 is a cross-sectional schematic drawing of a metal bonded distal end of an optical fiber bundle with an inner lumen according to embodiments of the invention;

FIG. 4 is a cross-sectional schematic drawing of a metal bonded distal end of an optical fiber bundle with an inner lumen and without a metal band according to embodiments of the invention;

FIGS. 5A-B show a cross-sectional view of a bonded metal distal tip and a side view of the distal region of fiber optic unit according to embodiments of the invention;

FIGS. 6A-B show a cross-sectional view of a glass-fused distal tip according to embodiments of the invention;

FIG. 7A shows a side-on view of a fiber optic unit with a monorail-type guidewire lumen according to embodiments of the invention;

FIG. 7B shows a cross-section of the distal tip of the fiber optic unit in FIG. 7A;

FIG. 8A shows a side-on view of a fiber optic unit with a central inner lumen according to embodiments of the invention;

FIG. 8B shows a cross-section of the distal tip of the fiber optic unit in FIG. 7A;

FIG. 9 is a flowchart showing selected steps in an method of making a metal bonded fiber optic bundle according to embodiments of the invention;

FIG. 10 is a flowchart showing selected steps in an method of making a fused-silica tip fiber optic bundle according to embodiments of the invention;

FIG. 11 is a flowchart showing selected steps in an method of making a hardened distal tip fiber optic bundle according to embodiments of the invention;

FIG. 12 is a flowchart showing selected steps in a method using a metal bonded fiber optic unit to ablate target tissue according to embodiments of the invention;

FIGS. 13A-B show SEM pictures of an aluminum bonded, swaged distal tip of a fiber optic unit after firing 1000 light pulses into a Plaster of Paris target;

FIGS. 14A-B show SEM pictures of an aluminum bonded, swaged distal tip of a fiber optic unit after firing 22,600 light pulses into a Plaster of Paris target;

FIGS. 15A-B show SEM pictures of an aluminum bonded, swaged distal tip of a fiber optic unit after firing 62,600 light pulses into a Plaster of Paris target;

FIG. 16 shows a pre-lase SEM picture of an individual optical fiber in a metal bonded, swaged distal tip of a fiber optic unit;

FIG. 17 shows an SEM picture of an individual optical fiber in a metal bonded, swaged distal tip of a fiber optic unit after firing 62,600 shots into a Plaster of Paris target;

FIGS. 18A-B show comparative SEM pictures of a 0.9 mm catheter tip having epoxy bonded optical fibers in pre-lased condition and after 1000 shots into a Plaster of Paris target; and

FIGS. 19A-B show comparative SEM pictures of a 1.7 mm catheter tip having epoxy bonded optical fibers in pre-lased condition and after 1000 shots into a Plaster of Paris target.

### DETAILED DESCRIPTION OF THE INVENTION

Fiber optic bundles having hard bonded distal tips are described. The tips may be part of a fiber optic unit that delivers pulses of light energy to ablate target tissue in a patient (e.g., a light-guiding catheter).

### Exemplary Optical Fiber Bundle Tips

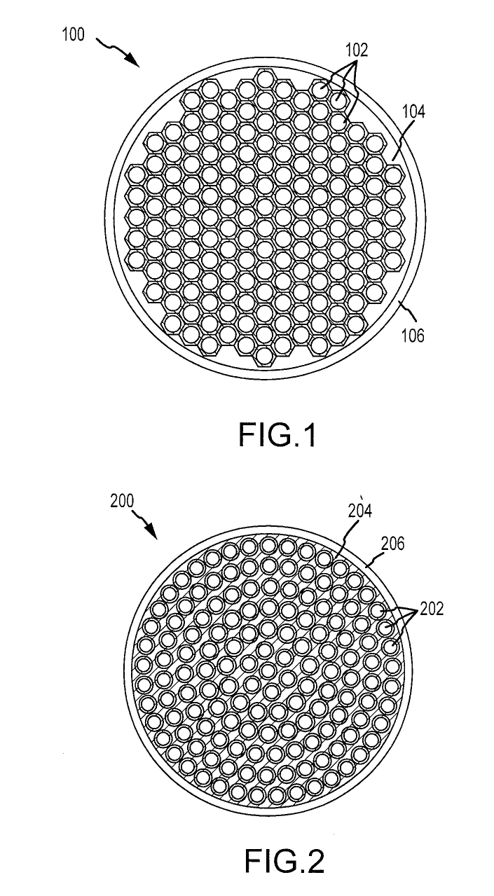

FIG. 1 shows a cross-sectional schematic drawing of an embodiment of one of these hard bonded distal tips 100 of an optical fiber bundle that uses metal as the bonding material. In this embodiment, metal coated distal ends of the optical fibers 102 form a solid cross-section at the distal end of the bundle. Each of the optical fibers is coated with a metal coating 104 at their distal ends, which are then swaged to bond the distal ends of fibers together. In the example shown, the optical fibers 102 are arranged at the distal end to form the metal coatings 104 into roughly hexagonal shaped interlocking units across the distal face of the bundle.

The metal used in the coatings may be a biocompatible metal or alloy of metals. For example, gold and platinum are known biocompatible metals that have been approved for use in medical devices that make direct contact with a patient's circulatory system. Thus, examples of the metals used in the coatings include gold, platinum, and alloys of the two metals. Additional metals may include silver, copper, and iron, among other metals.

The metal coatings 104 are formed on the cylindrical walls of the optical fibers starting at the distal ends of the fibers. They may extend about 1 mm to about 3 mm down the length of the fibers towards their proximal ends. The thickness of the metal coating 104 may be about 1 μm to about 30 μm. The optical fibers 102 themselves may be made from a light transmitting material such as quartz or bonded silica, and may have a diameter of about 50 μm.

The outer circumference of the distal tip of the unit may be surrounded by a metal band 106. The band may extend about 1 mm to about 3 mm down the length of the fibers towards their proximal ends, and may terminate with the metal coatings on the optical fibers 102. The band may be made from the same type of metal or alloy as the metal coating 104, or may be made from a different metal.

FIG. 2 shows a cross-sectional schematic of hardened-material bonded distal end of an optical fiber bundle 200 according to embodiments of the invention. The hardened-material used to bond the optical fibers in the distal tip may include glass, ceramic, or metal, among other materials.

The distal tip of the bundle is ringed by a band of radiopaque material 206 that adds additional integrity to the tip. The band may be made from the same hardened material that coats 204 the optical fibers 202, or from a different metal. For example, the band 206 may be made from a biocompatible metal or metal alloy such as gold and/or platinum. The thickness of the band 206 may be about 0.01 mm to about 0.02 mm (e.g., 0.012 mm).

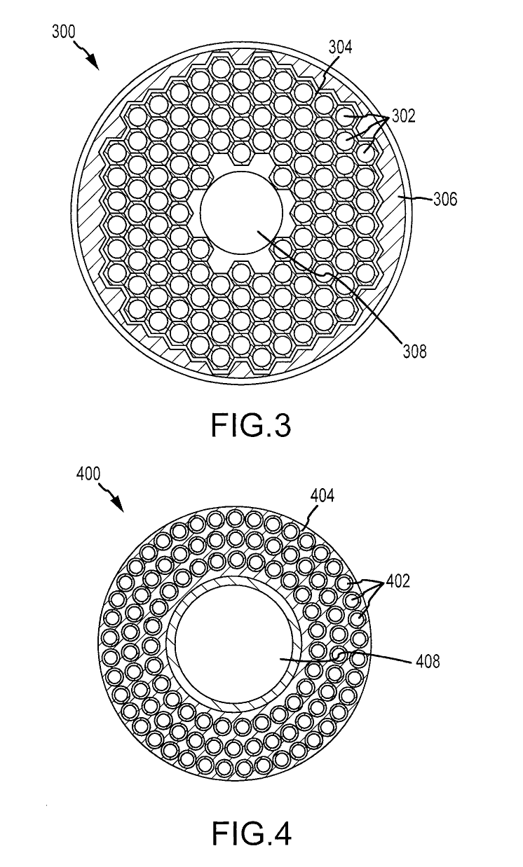

Referring now to FIG. 3, a cross-sectional schematic of a metal bonded distal end of an optical fiber bundle 300 with an inner lumen 308 and metal band 306 is shown. In this embodiment, the metal coated distal end of the optical fibers surround a lumen 308 at the center of the bundle. The lumen 308 may provide a passageway for fluid, a guidewire, or other equipment to travel through the fiber optic unit (e.g., a light guiding catheter) and out the distal end of the optical fiber bundle 300.

The metal bonded distal end shown in FIG. 3 is also ringed by a metal band 306. The metal band 306 may be made from the same or a different metal (or metals) that bond the individual fibers 302 together at the distal tip.

FIG. 4 shows another example of a metal bonded distal tip 400 with an inner lumen 408. This example shows an inner lumen formed around the center of the bundle surrounded by concentric rows of optical fibers 402 that are bonded together by the metal coatings 404 between each fiber 402. In this example, however, this is no metal band ringing the periphery of the bundle's distal tip.

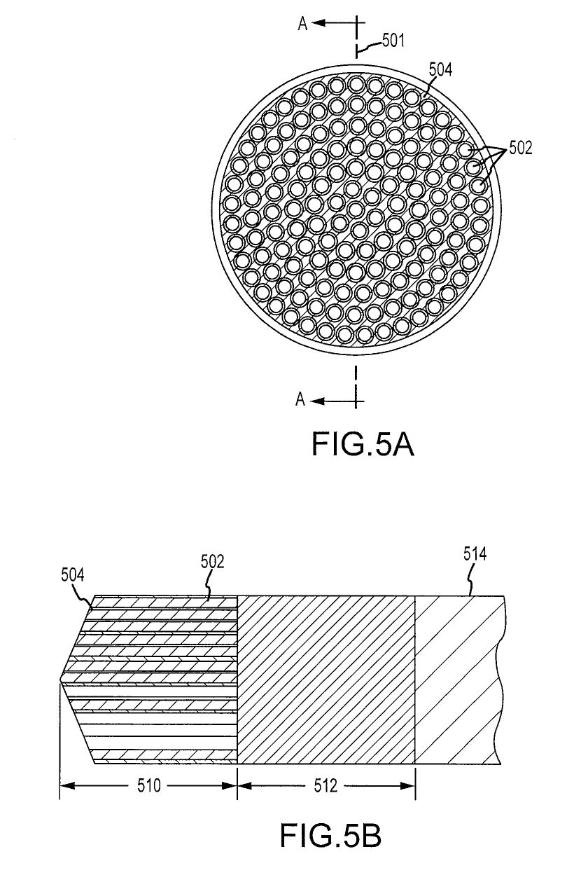

FIG. 5A shows a front cross-section of a metal bonded distal end of an optical fiber bundle similar to the one shown in FIG. 2, and FIG. 5B shows a side cross-sectional view of the same distal end cut along the A-A axis 501 in FIG. 5A. The side cross-section in FIG. 5B shows a plurality of optical fibers 502 separated by the bonded metal coating 504 between adjacent fibers 502. The metal bonded distal tip extends about 1 mm into the bundle, where it meets a protective coating 512 that is also about 1 mm deep. Beyond the metal bonded tip 510 and protective coating 512, the optical fibers 502 are individually coated with a polyamide coating 514 that reduces the chance that a fiber will fracture when twisting or bending. The polyamide coating 514 may extend to the proximal ends of the fibers, which are adapted to receive the light pulse from a light source and transmit it down the length of the fiber to the target tissue.

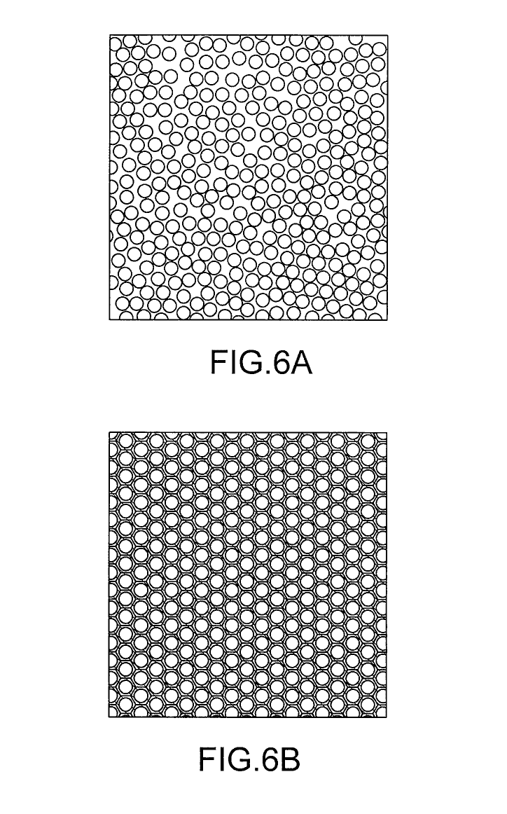

FIG. 6A shows a front cross-sectional views of the distal tips of an optical fiber bundle before the sidewalls for the optical fibers are fused together. FIG. 6B shows the same group of fibers after the distal tips have been hot fused together. Comparing the figures shows how the larger interstitial spaces and more random distribution of the tips is replaced with a more dense and ordered arrangement of fiber tips after they have been fused. The silica-fused tip of the optical fiber bundle can have an increased energy density of about 15% due to the shrinking of the inactive area between the fiber tips.

### Exemplary Fiber Optic Units

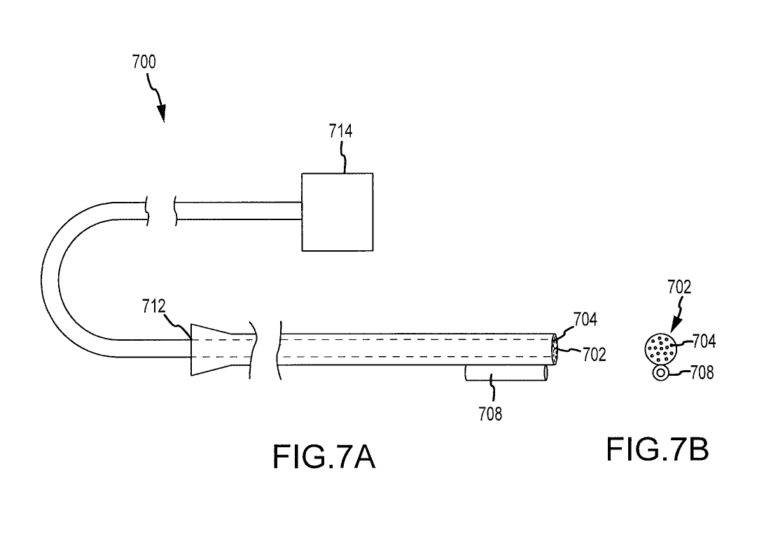

FIG. 7A is a pictorial drawing of a fiber optic unit 700 according to embodiments of the invention. The unit includes a bundle of optical fibers 702 whose distal ends are bonded by the hardened coating 704 on each of the fibers. These coatings may be made from glass, metal, or diamond-like-carbon (DLC), among other materials. The bundle distal tip has a solid cross-section (i.e., no internal lumen) and may have an external lumen 708 coupled to an exterior surface of the bundle. In this example, the exterior lumen is a “monorail” type design. The lumen is positioned proximate to the distal end of the fiber optic bundle and extends a fraction of the length of the bundle. A guidewire (not shown) may be inserted through the lumen and into a patient's vasculature until it reaches the target tissue. The lumen 708 and bundle are then advanced down the guidewire until the distal tip of the bundle is in position to ablate the target tissue. In additional examples, the external lumen 708 may extend a longer (or shorter) length of the bundle (e.g., the entire length of the bundle).

The proximal end of the optical fiber bundle 712 may be adapted to a light source 714 that generates the light energy pulses transmitted by the bundle. Examples of light sources include flash lamps and lasers. For example, the light source 714 may be an Excimer laser, such as a XeCl Excimer laser that outputs laser light pulses at about 308 nm. FIG. 7B shows a cross-sectional view of the distal tip 702 that includes the optical fibers 704 bundled together, and the external lumen 708 positioned below the tip 702. A band made from radiopaque material (not showns) may surround the distal tip 702.

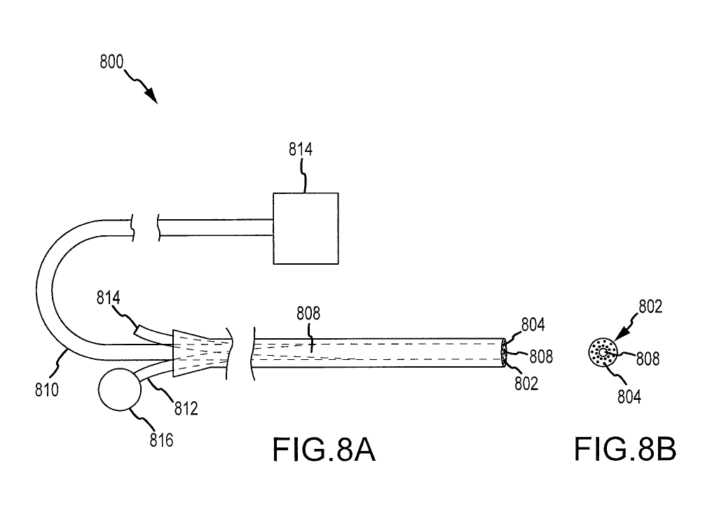

FIG. 8A shows is a pictorial drawing of a fiber optic unit 800 that is a light-guiding catheter according to embodiments of the invention. The catheter has a central inner lumen 808 that may be formed by the surrounding optical fiber bundle. The distal ends of the fibers 802 form an annular-shaped, hard tipped 804 bundle through which the light energy pulses exit into the target tissue. FIG. 8B shows a cross-section of the distal tip 802 with the central lumen 808 surrounded by the optical fibers 804.

The catheter embodiment shown has three proximal branches: The first branch 810 is adaptable to a light source 814 such as an Excimer laser. The second branch 812 is adaptable to a fluid source such as saline, a dye, or chemical solution to modify the tissue composition or absorption of light energy, among other fluids. For example, the second branch 812 may be coupled to a fluid pump 816 that pumps fluid from a fluid reservoir through the catheter lumen. The fluid can exits from the lumen's distal end to reach the area of the target tissue. When the fluid includes a dye, it may stain the target tissue to increase its absorption of the ablative light pulses. When the fluid includes a chemical solution, it may help dissolve hard components in the target tissue (e.g., calcium deposits). The pump action may also be reversed to vacuum ablated fragments of target tissue into the distal opening of the lumen.

The third proximal branch 815 is capable of receiving a tool (e.g., a guidewire) that gets advanced through the inner lumen. For example, the guidewire can be inserted into the third proximal branch, and advanced through the distal opening of the lumen and into a patient's body. When the distal end of the guidewire reaches a desired location inside the patient, the distal end of the catheter may be advanced over the guidewire until it is in position to ablate the target tissue.

### Exemplary Methods of Making Optical Fiber Bundle Tips

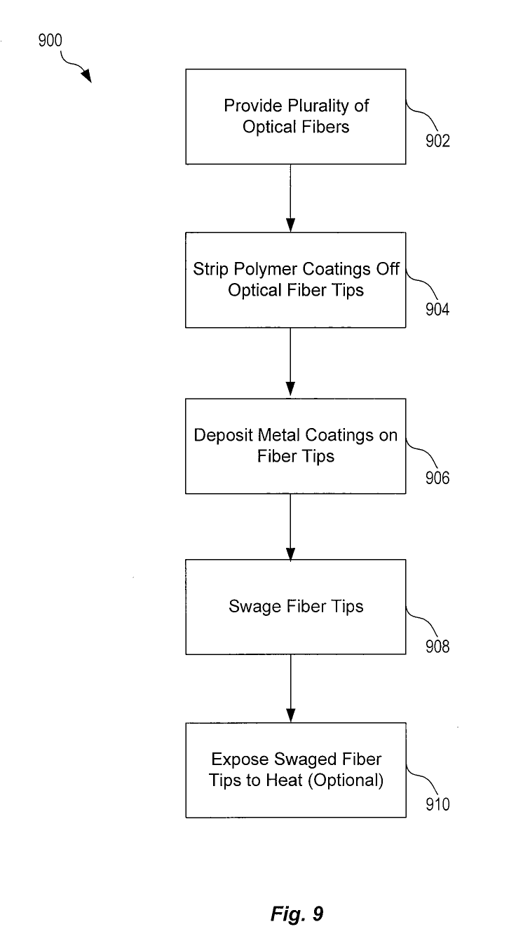

FIG. 9 shows steps in a method of making a fiber optic bundle 900 with a hardened metal bonded distal tip according to embodiments of the invention. The method may include providing a plurality of optical fibers 902, where the fibers can have a polymeric coating or jacket on the light transmitting fiber. The fiber may include a light transmitting core surrounded by a cladding layer. The core and cladding materials may include quartz, bonded silica, glass, and translucent plastics, among other materials. The polymeric coating material may be, for example, polyamide.

The polymeric coating is stripped from the distal ends of the optical fibers 904. The amount of coating that is stripped typically ranges from about 1 mm to about 3 mm of the length of the fiber. A metal coating may then be deposited on the cylindrical walls of the fiber tips 906. The coating may be deposited by physical vapor deposition (PVD)or chemical vapor deposition (CVD) of the metal on the exposed surfaces of the fibers, among other deposition methods. PVD processes may include metal sputtering (e.g., electron-beam sputtering, ion-beam sputtering, pulsed laser sputtering, etc.). CVD processes may include reacting and/or decomposing a gaseous or liquid precursor metal-organic precursor on the fiber optic surface to deposit the hardening material.

Fibers coated with metal may then be swaged to formed a metal bonded distal end of the optical fiber bundle 908. The swaging may be done by pressing the ends of the fibers in a tool or die. A metal band may be placed around the distal tip and swaged with the optical fibers. The swaging process puts more fiber in densely packed tip configuration, which allows the tip to deliver a higher energy light pulse to the target tissue. In some cases, the swaging alone is enough to bond the individual metal tips into the metal bonded bundle tip. In additional cases, heat may be applied during or after the swaging to aid in fusing of the bundle tip 910.

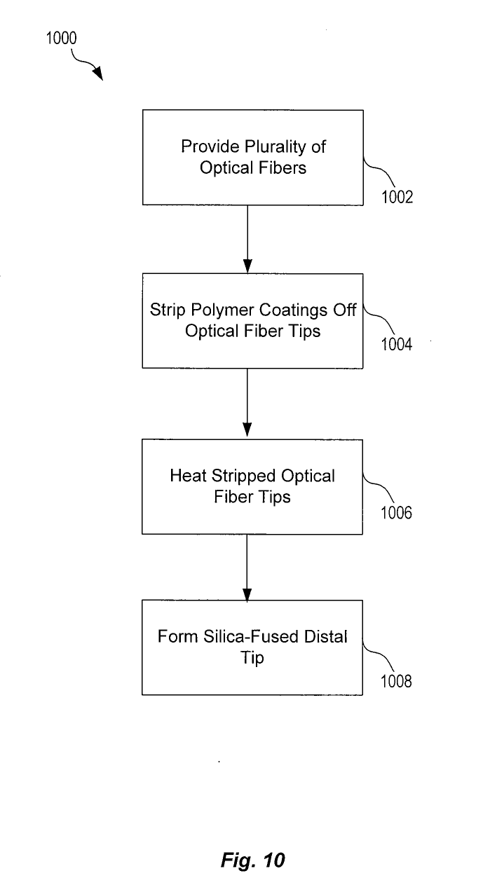

FIG. 10 is another a flowchart showing selected steps in an method 1000 of making a fused-silica tip fiber optic bundle according to embodiments of the invention. The method 1000 may include providing a plurality of optical fibers 1002 comprising a fused-silica light transmitting core surrounded with a polymeric coating. The polymeric coating may be stripped 1004 from the distal wall portions of the optical fiber, exposing the silica glass fibers underneath. The bare silica fiber tips may then be heated 1006 to fuse together the peripheries of the individual optical fibers to form the silica-fused distal tip 1008.

Optionally, a radiopaque band may be placed around the silica-fused distal tip. The radiopaque band may be made from a radiopaque material such as a metal. The metal may be a biocompatible metal or alloy such as gold or platinum, among other metals. The radiopaque band can help maintain the radiopacity of the catheter during imaging, as well as protect the optical fibers at the periphery of the bundle distal tip.

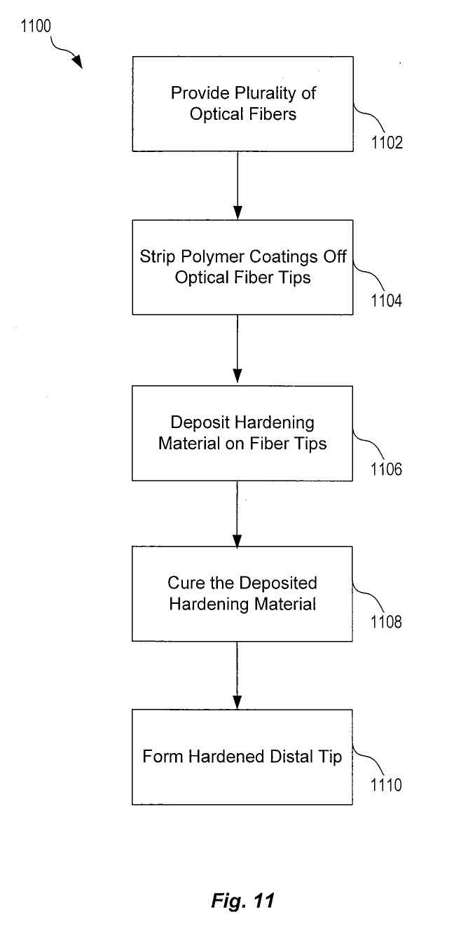

FIG. 11 is another flowchart showing selected steps in an method 1100 of making a hardened distal tip fiber optic bundle according to embodiments of the invention. The method 1100 may include providing a plurality of optical fibers 1102 that have a fused-silica light transmitting core surrounded with a polymeric coating. Polymer coatings on the distal ends walls of the optical fibers may be stripped from the fibers 1104, exposing the underlying distal tip fiber cores. A hardening material may then be deposited 1106 on the stripped fiber tips. This hardening material may be metal, silica glass, diamond-like-carbon, or some other type of hardening material. In the case of silica glass, glass stock material may be melted and applied to the fiber tips by, for example, dipping the distal tip into the molten glass. Another method may include the chemical vapor deposition of silica glass on the walls and interstitial spaces between the fibers from silicon-oxygen precursors (e.g., tetraethyl orthosilicate).

The deposited hardening material may then be cured 1108 to form the hardened distal tip 1110 of the optical fiber bundle. Curing methods may be as simple as cooling the tip to ambient temperature to allow the molten fused silica to solidify and harden. In additional embodiments, curing may involving a heating or annealing step following the chemical vapor deposition of silica on the fiber tips. Similar to the methods described above, a radiopaque band may be optionally formed around the hardened distal tip.

### Exemplary Methods of Using the Fiber Optic Units

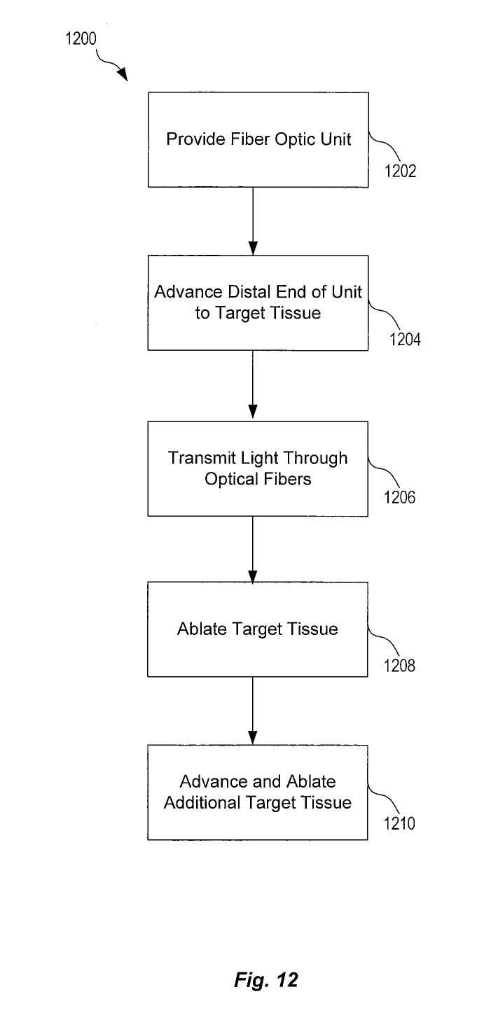

FIG. 12 is a flowchart showing selected steps in a method using a metal bonded fiber optic unit to ablate target tissue 1200 according to embodiments of the invention. The method may include providing a fiber optic unit 1202 that includes a bundle of optical fibers having a metal bonded distal end through which light pulses are transmitted to target tissue in a patient. As noted in the discussion of FIGS. 7 and 8 above, the fiber optic unit may include an external and/or inner lumen that can be used in conjunction with a guidewire to advance the distal end of the unit to a position proximate to the target tissue. For example, the guidewire may be inserted through the lumen and into the patient until the distal tip is placed in a treatment position with respect to the target tissue. This treatment position may have the distal tip of the guidewire placed proximate to, in contact with, or penetrating though the target tissue. When the guidewire is in position, the distal end of the fiber optic bundle may be advanced to the target tissue 1204 by sliding the lumen over the guidewire.

When the distal tip of the bundle reaches a position proximate to the target tissue (e.g., contacting the target tissue) light may be transmitted through the optical fibers and into the target tissue 1206. One or more pulses of transmitted light energy may be used to ablate a portion of the target tissue 1208. If necessary, the distal tip of the bundle may be further advanced toward remaining target tissue 1210 after a portion of the tissue has been ablated away. This process of ablating the target tissue and advancing the distal tip for further ablation may be repeated several cycles until the target tissue has been removed (or the vessel has been recanalized). The ablation light may be pulsed ultraviolet laser light generated by an Excimer laser (e.g., a XeCl Excimer laser lasing at around 308 nm).

### Experimental

Experiments were conducted to observe the durability of metal bonded optical fiber bundles and compare their durability to conventionally bundled tips bonded with epoxy. These experiments included firing pulses of XeCl Excimer laser light from bundled fiber optic units into an ex-vivo Plaster of Paris (i.e., calcium sulfate hemihydrate) target. The target is meant to simulate hard, calcified tissue like the types found in chronic total occlusions. The bonded distal ends of the fiber optic bundles are positioned proximate to the Plaster of Paris target and the XeCl Excimer laser is fired multiple times before an SEM image is made of the distal tip to examine any ablative damage caused by the acoustic shock and cloud of ablated target material.

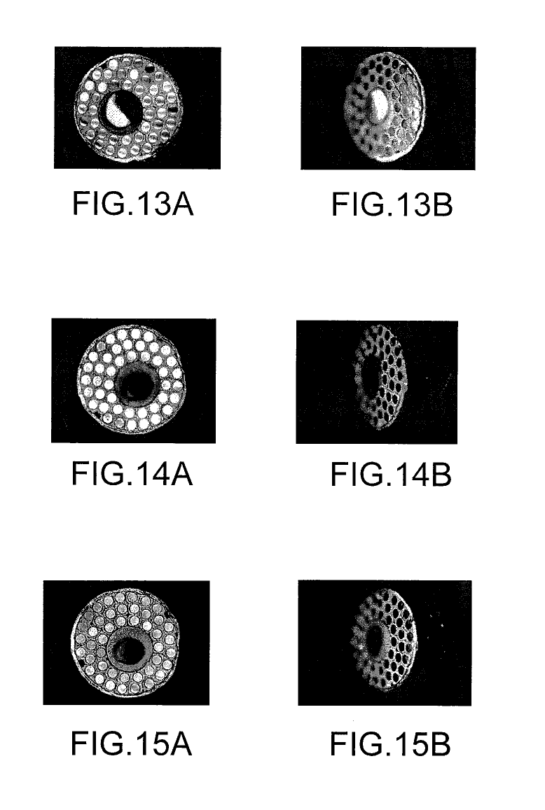

FIGS. 13A-B are SEM pictures of a 1.7 mm swaged, aluminum metal-bonded distal end of a fiber optic bundle after 1000 XeCl Excimer laser shots into a Plaster of Paris target. The laser was operating at 80% of peak power and firing at 80 FI and a rate of 80 Hz. As FIGS. 13A&B show, the ablative damage is minimal in the interstitial aluminum metal areas between the ends of the optical fibers. FIGS. 14A&B show additional SEM pictures of the distal end of the tip after 22,600 laser shots with again little noticeable damage to the aluminum. The durability test of the aluminum swaged bundle tip was further extended to 62,600 shots in FIGS. 15A&B which shows an aluminum tip that still has excellent integrity in the metal spaces between the optical fibers.

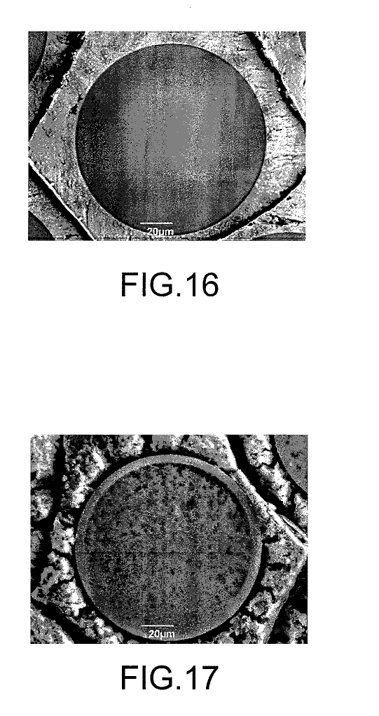

FIGS. 16 and 17 show higher-magnification SEM pictures of an individual optical fiber surrounded by the aluminum metal fusing material pre-lase (FIG. 16) and after 62,600 Excimer laser shots into a Plaster of Paris target (FIG. 17). Some cracking and separation between the metal and fiber optic tip is visible after 62,600 shots, but even after this large number of shots, the metal has not significantly receded in distal tip region of the fiber.

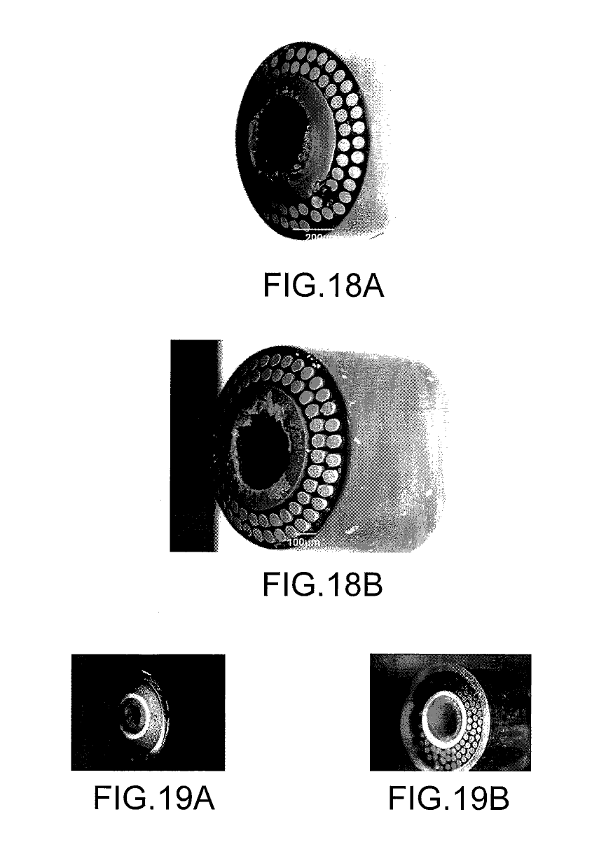

In comparison, FIGS. 18A&B show comparative SEM pictures of a conventional 0.9 mm catheter tip having epoxy bonded optical fibers. Similar to the pictures of the aluminum bonded bundles above, the epoxy bonded tip has a central lumen surrounded by an annular shaped ring of optical fibers. In the case of this bundle, the optical fibers are arranged in an inner and outer concentric row of optical fibers. FIG. 18A shows the epoxy bonded optical fiber tip before any laser pulses have been fired through the tip, and FIG. 18B shows the tip after 1000 laser pulses from an XeCl Excimer laser have been fired into a proximate Plaster of Paris target.

FIG. 18B shows that the epoxy has recessed significantly from the distal end of the catheter tip after 1000 laser shots. The receding epoxy exposes the distal ends of the optical fibers, making them prone to wear and fracture that can reduce their light transmitting efficiencies.

A similar effect is seen in FIGS. 19A&B, which show comparative SEM pictures of a 1.7 mm catheter tip having epoxy bonded optical fibers in pre-lased condition and after 1000 shots into a Plaster of Paris target. The pre-lase tip shows 353ND expoxy formed up to the distal tips of the optical fibers. After 1000 shots of a XeCl Excimer laser however, the epoxy has significantly receded behind the distal ends of the fiber optic tips. Thus, for both of the comparative examples shown in FIGS. 19A&B the disintegration of the epoxy at 1000 laser shots has progressed substantially further than the disintegration of the interstitial aluminum after 62,600 shots.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the optical fiber” includes reference to one or more optical fibers and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

1. A fiber optic unit to ablate tissue with light, the unit comprising:
a bundle of optical fibers having a bundle proximal end adaptable to a light source and a bundle distal end though which the light exits to reach the tissue;
a metal coating formed around distal ends of each of the optical fibers, wherein the metal coatings are swaged to bond the distal ends of the optical fibers together.

2. The fiber optic unit of claim 1, wherein the unit includes a lumen with an opening at the bundle's distal end.

3. The fiber optic unit of claim 2, wherein the lumen is centered in a middle of the bundle's distal end and surrounded by the optical fibers.

4. The fiber optic unit of claim 2, wherein the lumen extends from the bundle's proximal to the bundle's distal end.

5. The fiber optic unit of claim 2, wherein the unit comprises a guidewire that can be inserted into the lumen.

6. The fiber optic unit of claim 2, wherein the lumen is adaptable to a fluid source that can supply a fluid to flow through the lumen.

7. The fiber optic unit of claim 6, wherein the fluid comprises a solution to modify the absorption of the light by the tissue.

8. The fiber optic unit of claim 6, wherein the fluid comprises saline and a tissue dissolving agent.

9. The fiber optic unit of claim 1, wherein the metal coating comprises a biocompatible metal or metal alloy.

10. The fiber optic unit of claim 1, wherein the metal coating is selected from the group consisting of aluminum, gold, and platinum.

11. The fiber optic unit of claim 1, wherein a metal band surrounds the bundle's distal end.

12. The fiber optic unit of claim 11, wherein the metal band comprises the same material as the metal coatings on the optical fibers.

13. The fiber optic unit of claim 11, wherein the metal band comprises gold or platinum.

14. The fiber optic unit of claim 1, wherein the light source comprises a 308 nm Excimer laser.

15. The fiber optic unit of claim 1, wherein the metal coatings formed around the distal ends of the optical fibers extend about 1 mm to about 3 mm down the length of the fibers.

16. The fiber optic unit of claim 1, wherein the distal end of the bundle is about 0.5 to about 2 mm in diameter.

17. A method of making an optical fiber bundle for tissue ablation having a metal bonded distal end, the method comprising:
providing a plurality of optical fibers comprising a light transmitting core and a polymeric coating;
stripping the polymeric coating from distal wall portions of the optical fiber;
depositing a metal coating on the stripped distal wall portion of the optical fiber; and
swaging the distal ends of the optical fibers together to form the metal bonded distal end of the bundle.

18. The method of claim 17, wherein the light transmitting core comprises quartz or bonded silica.

19. The method of claim 17, wherein the polymeric coating comprises polyamide.

20. The method of claim 17, wherein the step of depositing the metal coating comprises metal vapor deposition of the coating on the distal walls.

21. The method of claim 20, wherein the metal coating comprises gold or platinum.

22. The method of claim 17, wherein the method further comprises placing a metal band around the distal end of the optical fiber bundle.

23. The method of claim 17, wherein the distal ends of the optical fibers are swaged around a distal end of a lumen.

24. A method to ablate target tissue with light, the method comprising:
providing a fiber optic unit comprising a bundle of optical fibers having a bundle proximal end adapted to a light source and a bundle distal end though which the light exits to reach the tissue, wherein metal coatings formed around distal ends of each of the optical fibers are swaged to bond the distal ends of the optical fibers together;
advancing the bundle's distal end to a position proximate to the target tissue; and
transmitting the light through the optical fibers to ablate the target tissue.

25. The method of claim 24, wherein the target tissue comprises a calcified vascular occlusion.

26. A fiber optic unit to ablate tissue with light, the unit comprising:
a bundle of optical fibers having a bundle proximal end adaptable to a light source and a bundle distal end though which the light exits to reach the tissue;
a glass coating formed around distal ends of each of the optical fibers, wherein the glass coatings are fused to bond the distal ends of the optical fibers together.

27. The fiber optic unit of claim 26, wherein a radiopaque band surrounds the bundle's distal end.

28. The fiber optic unit of claim 27, wherein the radiopaque band comprises a metal.

29. A method of making an optical fiber bundle for tissue ablation having a glass fused distal end, the method comprising:
providing a plurality of optical fibers comprising a fused-silica light transmitting core surrounded with a polymeric coating;
stripping the polymeric coating from distal wall portions of the optical fiber; and
heating the stripped distal ends of the optical fibers to fused them together into the glass fused distal end of the optical fiber bundle.

30. The method of claim 29, wherein the method further comprises forming a radiopaque band around the glass fused distal end of the optical fiber bundle.

31. The method of claim 30, wherein the radiopaque band comprises a metal.

32. The method of claim 31, wherein the metal band is formed by depositing metal vapor on the glass fused distal tip.

33. A method of making an optical fiber bundle for tissue ablation having a hardened distal end, the method comprising:
providing a plurality of optical fibers comprising a fused-silica light transmitting core surrounded with a polymeric coating;
stripping the polymeric coating from distal wall portions of the optical fiber;
depositing a hardening material on the stripped distal ends of the optical fibers; and
curing the deposited hardening material to form the hardened distal end of the optical fiber bundle.

34. The method of claim 33, wherein the depositing of the hardening material comprises applying melted glass to the stripped distal ends of the optical fibers.

35. The method of claim 33, wherein the depositing of the burdening material comprises vapor depositing a diamond-like carbon film on the stripped distal ends of the optical fibers.

36. The method of claim 33, wherein the method further comprises forming a radiopaque band around the hardened distal end of the optical fiber bundle.
